(19) United States
(12) Patent Application Publication
(10) Pub. No.: US 2025/0302525 A1
(43) Pub. Date: Oct. 2, 2025

(54) INNER SHAFT, MANUFACTURING METHOD, AND RESECTOSCOPE

(71) Applicant: KARL STORZ SE & CO. KG, Tuttlingen (DE)

(72) Inventors: Rainer HERMLE, Tuttlingen (DE); Uwe WITTKE, Tuttlingen (DE)

(21) Appl. No.: 18/865,882

(22) PCT Filed: May 15, 2023
(86) PCT No.: PCT/EP2023/063031

§ 371 Date: Nov. 14, 2024

(30) Foreign Application Priority Data
May 17, 2022 (DE) 10 2022 112 285.9

(51) Int. Cl. A61B 18/14; A61B 18/00

(52) U.S. Cl. CPC A61B 18/149; A61B 2018/00517; A61B 2018/00601

(57) ABSTRACT

An inner shaft for a working element of a resectoscope includes a distal end portion and at least three spacer elements which are arranged on an outer surface of the inner shaft. The spacer elements are distributed at different positions in the region of the distal end portion such that the inner shaft can be guided and/or placed within an outer shaft at a distance from the outer shaft in a predefined way at least in the distal end portion. A method produces the inner shaft and a resectoscope is provided with the inner shaft.

### CROSS REFERENCE TO RELATED APPLICATIONS

This application is a United States National Phase Application of International Application PCT/EP2023/063031, filed May 15, 2023, and claims the benefit of priority under 35 U.S.C. § 119 of German Application 10 2022 112 285.9, filed May 17, 2022, the entire contents of which are incorporated herein by reference.

### FIELD OF THE INVENTION

The present invention relates to an inner shaft for a working element of a resectoscope. The invention also relates to a method for producing an inner shaft and to a resectoscope having an outer shaft and such an inner shaft.

### TECHNICAL BACKGROUND

Such Surgical instruments are used for different applications. For example, they can be used in the area of minimally invasive surgery and have a surgical tool. Surgical instruments can be configured as resectoscopes and have a working element that is configured, for example, to guide the surgical tool, such as an electrode.

Resectoscopes are known in particular for urological or gynecological applications. The working element with the electrode can be inserted into the patient's body through the urethra to a surgical site. At one distal end (i.e., remote from the user), for example, the electrode has a cutting loop that can be used to ablate tissue. The ablated tissue can be removed again through a drain channel by means of an irrigation liquid that is guided through an irrigation channel or inflow channel to the surgical site. The inflow and drain channels allow for continuous rinsing, which also serves to keep a viewing window clear to ensure a perfect endoscopic view at all times.

DE 100 56 618 A1 describes a double-shaft endoscope for continuous irrigation, comprising an inner shaft which encloses an inflow channel and in which an optical unit is accommodated, and comprising an outer shaft which surrounds the inner shaft such that a drain channel is formed. The inner shaft accommodates a carrier of a resection loop movable in the axial direction. The inner shaft and the outer shaft are arranged such that they can be moved relative to each other.

To guide the tool, for example the electrode, the surgical instrument generally has a carriage which is supported for movement along a longitudinal axis of the surgical instrument and which is connected to the surgical tool. The carriage can be moved by means of a handle or with an actuator that can be operated via a robot interface.

If an electrode is inserted into the working element, the cutting loop of the electrode can be moved in the axial direction by moving the carriage axially-for example, to ablate tissue. It is necessary that the electrode can be moved relative to the outer shaft. Therefore, the working element with the electrode must be guided at a distance from the outer shaft.

In known embodiments, a spacer element is usually provided in the region of the handle, for example in the region of an electrode clamp, the spacer element being arranged on the working element so as to protrude downwards and being in contact with an inner contour of the outer shaft. This spacer element at the proximal end allows the electrode to be held at a distance from the outer shaft at the distal end.

A disadvantage is that the spacer element arranged at the proximal end creates friction when the electrode is axially moved, this friction being perceived as disturbing during operation of the surgical instrument.

Another disadvantage is that the working element is supported in a region that is remote from the distal end at which the electrode is arranged. Therefore, lowering over the length of the working element up to the electrode relative to the outer shaft may occur.

However, in order to ensure safe and reliable desired usability, it is desirable to always allow the electrode to be returned into the inner shaft.

### SUMMARY OF THE INVENTION

Against this background, the present invention is based on the object of specifying improved support of the electrode relative to the inner shaft or relative to the outer shaft.

According to the invention, this object is achieved by means of an inner shaft having features according to the invention, by means of a method having features according to the invention, and by means of a resectoscope having features according to the invention.

Accordingly, the following are provided:
An inner shaft for a working element of a resectoscope, comprising a distal end portion and at least three spacer elements which are arranged on an outer surface of the inner shaft, wherein the spacer elements are distributed at different positions in the region of the distal end portion such that the inner shaft can be guided and/or placed within an outer shaft at a distance from the outer shaft in a predefined way at least in the distal end portion.
A method for producing an inner shaft for a working element of a resectoscope, wherein the form of the inner shaft is produced by erosion.
A resectoscope comprising an outer shaft and an inner shaft, wherein the inner shaft is guided in the outer shaft in such a way that, in the region of a distal end portion, lowering of the working tool, in particular an electrode, relative to the outer shaft is prevented by the spacer elements.

The finding underlying the present invention is that a lowering of the electrode can be prevented by guiding the working element at the distal end of the surgical instrument.

The idea underlying the present invention is to guide the inner shaft in a region at the distal end by means of at least two spacer elements in point or linear contact with the outer shaft.

Lowering of the electrode at the distal end can be prevented by the spacer elements, since the distance between the inner shaft and the outer shaft can be directly set and maintained by the spacer elements in the region of the distal end portion, even when the electrode or the working element is axially moved. Advantageously, the working element is therefore not arranged freely floating in the region of the distal end, but rather is guided on the inner shaft. This ensures that, when the electrode is axially moved, it can be moved back into the outer shaft. This advantageously means that there is no risk of the extended electrode being lowered so far due to gravity that it becomes impossible to move back. This means that the electrode can be easily retracted into the outer shaft at any time.

Furthermore, by guiding the working element on the inner shaft, contact between the working element and the outer shaft can be prevented, which means that no friction that disrupts the working process results when the electrode is retracted and extended. When the working element is axially moved, no frictional resistance occurs between the working element and the outer shaft, since there is no direct contact between these two elements. Rather, the working element is guided on the inner shaft and is thus held at a distance from the outer shaft, even in the region of the distal end.

The inner shaft can have different designs. For example, the inner shaft may have a discontinuous cross section, wherein convex and/or concave bulges can adjoin straight portions. In particular, the cross section is symmetrical in a plane or an axis, so that the working element can be guided symmetrically on the cross section.

Preferably, the cross section has a small wall thickness, in particular 0.09 to 0.20 mm, for example approximately 0.15 mm. The wall thickness can vary in the region of the cross section. The inner shaft can also be configured with a constant cross section all the way around.

A spacer element is an element that protrudes from the outer surface of the inner shaft. Thus, the inner shaft can be guided at a distance from the outer shaft by the spacer element. In particular, the at least two spacer elements are dimensioned such that a distance from the outer shaft can be created around the entire periphery of the inner shaft. This allows a peripheral drain channel to be formed between the inner shaft and the outer shaft. Since the spacer elements do not run over the entire length of the inner shaft, the drain channel is disturbed by the spacer elements only in a very short axial portion, and the returning medium can spread in the axial direction in front of or behind each spacer element without being hindered.

The spacer elements are preferably rounded so that no damage occurs when they are in contact with the outer shaft.

The distal end portion is preferably understood to be a region that is small in relation to the entire length of the inner shaft. Preferably, the distal end portion is adjacent to the distal end of the inner shaft.

The method of erosion is suitable in particular for thin wall thicknesses, such as are used in the inner shaft. A wall thickness between 0.09 and 0.2 mm can be considered a thin wall thickness. In particular, the wall thickness is between 0.1 and 0.17 mm. Furthermore, very high precision can be achieved through wire erosion.

Advantageous embodiments and developments are shown in the further dependent claims and in the description with reference to the figures in the drawing.

According to an advantageous embodiment, the spacer elements can be arranged on the outer surface of the inner shaft at different peripheral positions, in particular with a mutual offset of at least 90°, preferably with a mutual offset of more than 100°. This allows the inner shaft to be reliably held at a distance from the outer shaft all around. Preferably, two such spacer elements are identically shaped so that the inner shaft can be held symmetrically with respect to the outer shaft at least in one plane.

According to an advantageous embodiment, the spacer elements can be arranged at different positions along a longitudinal axis of the inner shaft. For example, one spacer element may be arranged axially closer to the distal end than another spacer element is. The spacer elements can also be formed with different axial lengths.

According to an advantageous embodiment, the spacer elements can be formed integrally with the inner shaft. The spacer elements can, for example, be configured as bulges with an open or hollow cross section on the inside. The spacer elements can also be made of solid material.

According to an advantageous embodiment, at least one spacer element can be dome-shaped and at least one spacer element can be bead-shaped, wherein the bead-shaped spacer element extends over a larger longitudinal portion along the inner shaft in comparison with the dome-shaped spacer element. Advantageously, the dome-shaped spacer element is formed by a bulge. For example, the dome-shaped spacer element may be arranged closer to the distal end than the bead-shaped spacer element is. The term “bead-shaped” is understood to mean, in particular, an elongate form oriented in the axial direction of the inner shaft. This form can be wedge-shaped in cross section, for example configured with a cross section that is triangular at least in portions, and/or integral with the inner shaft.

According to an advantageous embodiment, two bead-shaped spacer elements located opposite in cross section can be provided, wherein in particular the dome-shaped spacer element is arranged centrally with respect thereto. This can reliably ensure a distance between the inner shaft and the outer shaft all around the periphery.

According to an advantageous embodiment, the inner shaft can be formed, in at least one region, for contacting at least one working tool, wherein the working tool can be guided at a distance from the outer shaft due to the shape of the cross section at a predefined position. The region is understood to be a portion of the cross section of the inner shaft which in particular is adapted to the shape of the working element. Contact between the inner shaft and the working element preferably allows the working element to be kept at a distance from the outer shaft. For example, a sort of retainer for the working element can be formed by virtue of the shape of the inner shaft in the region. The working element is preferably immovably held in this retainer. The working element is preferably held outside, i.e., on an outer surface, on the inner shaft.

According to an advantageous embodiment, the inner shaft can form a working channel for guiding and/or fixing a working tool, the working channel being arranged within a cross section of the inner shaft. The working channel can be formed by one or two elements that are at least in portions semicircular or shell-shaped. This allows another working tool to be guided, in particular held in a clamping manner, at a distance from the outer shaft.

According to an advantageous embodiment, the spacer elements and the at least one region for contacting the at least one working tool can be arranged adjacent to each other on the inner shaft. As a result, the working element can be held outside, i.e., on an outer surface, on the inner shaft, while a spacer element is also arranged on an outer surface of the inner shaft. Thus, the diameter of the outer shaft can be reduced and a surgical instrument of very small diameter can be provided.

According to an advantageous embodiment, the outer surface can be concave in portions and convex in portions in different peripheral regions. In this way, the region for clamping or holding the working element can be formed and at the same time a relatively large inner surface for forming an inflow channel can be maintained.

According to an advantageous embodiment, a wall thickness of the inner shaft can be, at least in portions, 0.1 mm to 0.2 mm, in particular approximately 0.15 mm. This makes it possible to provide a very finely structured surgical instrument.

In particular, the inner shaft has a constant cross section over the length. The spacer elements are an exception to this; in particular, they are arranged only in the region of the distal end portion.

According to an advantageous embodiment of the method, at least one spacer element can be formed by an embossing process. In this case, the spacer element can be subsequently formed onto an inner shaft which has already been produced. Furthermore, weight and material can be saved.

Advantageously, the form of the inner shaft and the spacer elements are able to be produced by an additive process. For example, laser sintering can be used to implement metal 3D printing.

According to an advantageous embodiment of the resectoscope, the working tool, in particular the electrode, can be positioned and guided, in particular can be returned after moving out, without contact with the outer shaft. This is achieved by the spacer elements in the region of the distal end portion, wherein lowering of the electrode or of the working element at the distal end is prevented. In particular, guiding of the working element by the geometry of the inner shaft, i.e., support of the working element on the inner shaft, can prevent the electrode from being lowered relative to the inner shaft at the distal end.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

The present invention is explained in greater detail below with reference to the exemplary embodiments shown in the schematic figures of the drawing. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view showing a working element with an electrode from the prior art;

FIG. 2 is a partial perspective view showing a distal end portion of an inner shaft according to one embodiment;

FIG. 3 is a cross sectional view taken through a distal end portion of a surgical instrument; and

FIG. 4 is a cross sectional view taken through a distal end portion of an inner shaft.

The accompanying figures of the drawing are intended to provide a further understanding of the embodiments of the invention. They illustrate embodiments and, in conjunction with the description, serve to explain principles and concepts of the invention. Other embodiments and many of the advantages mentioned are shown in the drawings. The elements in the drawings are not necessarily shown to scale.

In the figures of the drawing, identical, functionally equivalent, and identically acting elements, features, and components are each provided with the same reference signs, unless otherwise specified.

### DESCRIPTION OF PREFERRED EMBODIMENTS

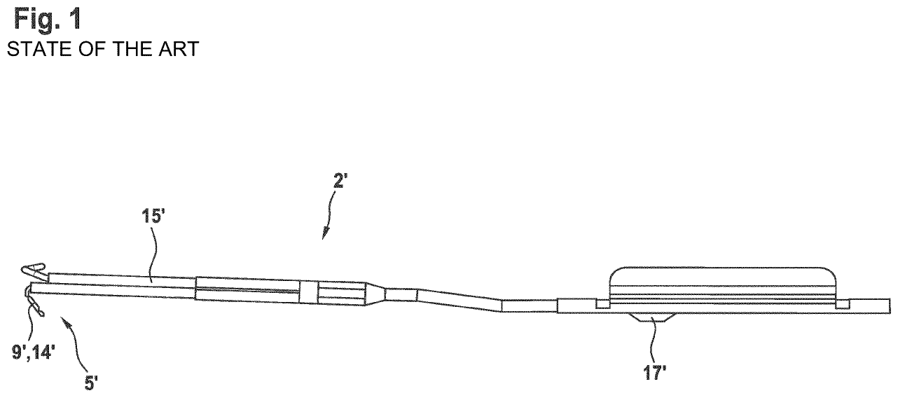

Referring to the drawings, FIG. 1 shows a working element 2′ from the prior art. A working tool 9′ is arranged in a distal end portion 5′. The working tool is in the form of an electrode 14′. The electrode 14′ is guided in an electrode tube 15′. At the proximal end portion opposite from the distal end portion 5′, an electrode clamp is arranged on the working element 2′. At the bottom of the electrode clamp, a spacer element 17′ is provided, which is contacted with an inner contour of an outer shaft (not shown) when installed in a surgical instrument. This allows the working element 2′ to be held at a distance from the outer shaft, thereby preventing contact between the electrode and the outer shaft. A disadvantage is that friction occurs between the outer shaft and the spacer element 17′ when the electrode is axially moved in the surgical instrument. Furthermore, the electrode 14′ and the working element 2′ are arranged freely floating in the region of the distal end portion 5. The electrode 14′ can therefore be lowered unintentionally due to gravity alone. As a result, a return of the electrode 14′ into the outer shaft cannot always be ensured.

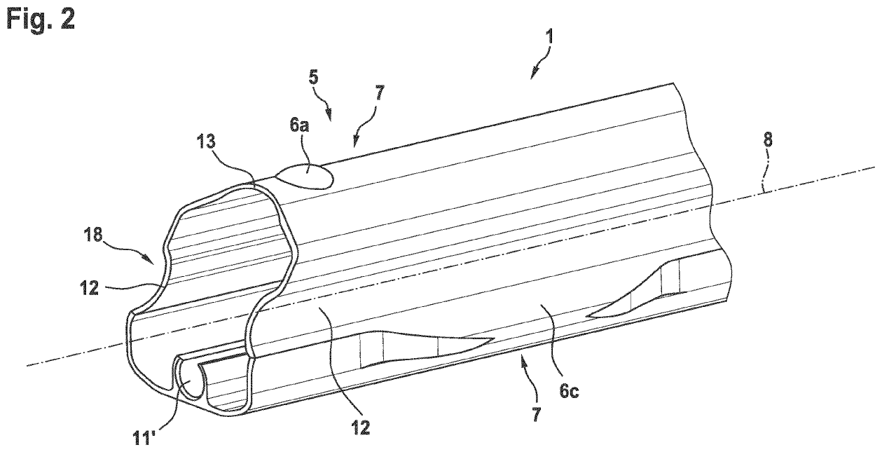

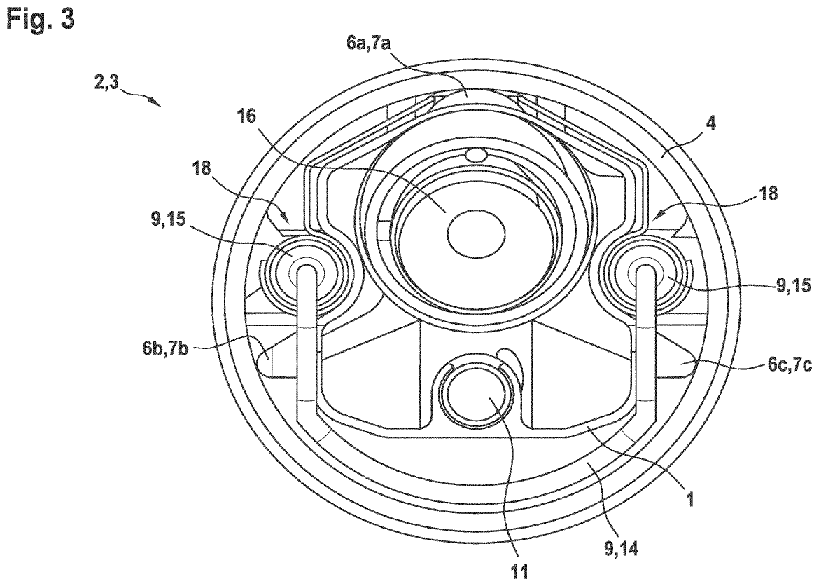

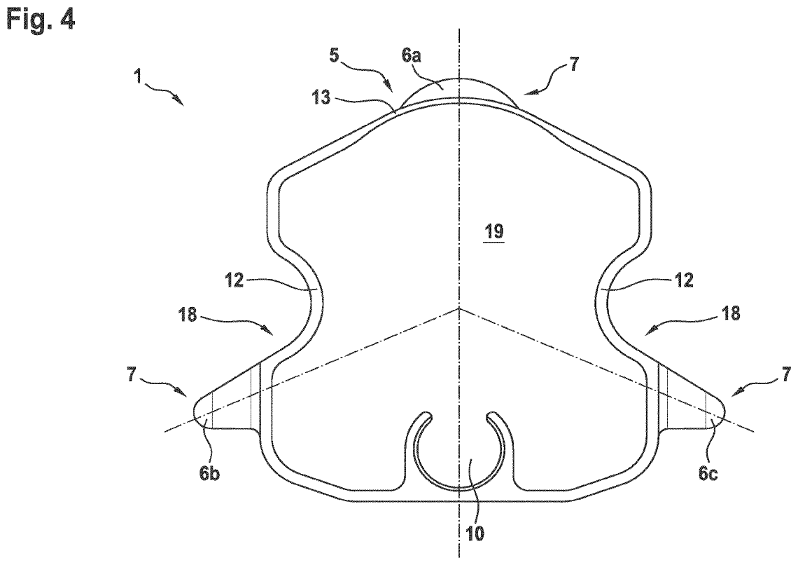

FIG. 2 shows a distal end portion 5 of an inner shaft 1 according to one embodiment. In contrast to the embodiment of FIG. 1, at the distal end portion 5 three spacer elements 6a to 6c are arranged at three different peripheral positions 7 on an outer surface of the inner shaft 1. Each spacer element 6a to 6c is placed at only a slight distance from the distal end. The spacer element 6a is located closer to the distal end than the spacer element 6c is. Another spacer element 6b (shown in FIGS. 3 and 4) is formed identically to the spacer element 6c and arranged identically.

In this embodiment, the spacer element 6a is arranged centrally in the upper region of the inner shaft 1 and is dome-shaped. The spacer element 6a can be produced, for example, by an embossing process. The other visible spacer element 6c is formed in a bead shape along the longitudinal axis 8 of the inner shaft 1. It can be produced, for example, by erosion during the production of the inner shaft 1.

The illustrated spacer elements 6a and 6c are arranged on the inner shaft 1 in such a way that the inner shaft 1 can be arranged at a distance from an outer shaft (not shown) all around. In this embodiment, the spacer element 6c is formed with a wedge-shaped cross section. All the spacer elements 6a to 6c are rounded in a region that comes into contact with the outer shaft, so that damage to the outer shaft can be prevented.

FIG. 3 shows a cross section through a distal end portion 5 of a surgical instrument. The electrode 14 is guided in an electrode tube 15. The electrode tube 15 is held in a region 18 by the cross-sectional shape of the inner shaft 1 in such a way that lowering of the electrode 14 and of the electrode tube 15 can be prevented at the distal end portion 5, in particular over the entire length of the working element 2. Thus, a return of the electrode 14 into the outer shaft 4 can be ensured at all times. The working tool 9 is held, in particular clamped, in a concave peripheral region 12 of the inner shaft 1, while the spacer elements 6a to 6c are each arranged on a convex peripheral region 13 or a straight peripheral region.

The spacer elements 6a to 6c are arranged at different peripheral positions 7a to 7c at a distance from each other. The spacer elements 6a to 6c are in particular placed offset from each other by at least 90°, preferably at least 100°.

In the cross-sectional illustration, it is clear that the spacer elements 6a to 6c are arranged adjacent to the electrode tube 15 on the inner shaft 1. As a result, the inner shaft 1 can be held at a distance from the outer shaft 4 all around, while at the same time the smallest possible diameter of the outer shaft 4 can be ensured.

In FIG. 3, an optical element 16 is also held on the inner shaft 1 by virtue of the shape of the inner shaft 1. The regions 18 and an upper convex peripheral region 13 serve as clamping portions; see also FIG. 2. Furthermore, a working tool 11 is held in a clamping manner in the inner shaft 1 in lower region of the inner shaft 1 by means of shell-shaped elements. This allows another working channel to be formed.

FIG. 4 shows a cross section through a distal end portion 5 of an inner shaft 1. The position of the spacer elements 6a to 6c with a mutual offset of at least 90° around the periphery of the inner shaft 1 can be seen. A distance between the spacer elements 6b and 6c is greater than a distance of each from the spacer element 6a. In this embodiment, the inner shaft 1 is also symmetrical with respect to an axis (here a vertical axis). This allows the working element with the electrode tube to be held or placed symmetrically on the inner shaft in the regions 18.

An inflow channel 19 is formed inside the inner shaft 1. The drain channel is formed between the inner shaft 1 and the outer shaft 4; see FIG. 3.

Although the present invention has been fully described above with reference to preferred exemplary embodiments, it is not limited thereto, but can be modified in a variety of ways.

For example, the number of spacer elements 6 may differ from the number shown. Furthermore, the spacer elements 6 can be arranged at different peripheral positions 7. Two or three dome-shaped spacer elements 6 are also conceivable. In another embodiment, all the spacer elements 6, for example three or four spacer elements 6, can be bead-shaped.

Furthermore, the spacer elements 6 can be directly adjacent to the distal end or can all be arranged at an equal distance from the distal end. It is only important that lowering of the electrode directly in the region of the distal end is prevented by the at least two spacer elements 6.

While specific embodiments of the invention have been shown and described in detail to illustrate the application of the principles of the invention, it will be understood that the invention may be embodied otherwise without departing from such principles.

### List of Reference Characters

1 Inner shaft
2 Working element
3 Resectoscope
4 Outer shaft
5 Distal end portion
6 Spacer element
7 Peripheral position
8 Longitudinal axis
9 Working tool
10 Working channel
11 Working tool
12 Concave peripheral region
13 Convex peripheral region
14 Electrode
15 Electrode tube
16 Optical element
17 Spacer element on the electrode clamp
18 Region
19 Inflow channel

1. An inner shaft for a working element of a resectoscope, the inner shaft comprising:
a distal end portion; and
at least three spacer elements which are arranged on an outer surface of the inner shaft, wherein the spacer elements are distributed at different positions in a region of the distal end portion such that the inner shaft can be guided and/or placed within an outer shaft at a distance from the outer shaft in a predefined way at least in the distal end portion.

2. The inner shaft according to claim 1, wherein the spacer elements are arranged on the outer surface of the inner shaft at different peripheral positions, with a mutual offset of at least 90°.

3. The inner shaft according to claim 1, wherein the spacer elements are arranged at different positions along a longitudinal axis of the inner shaft.

4. The inner shaft according to claim 1, wherein the spacer elements are formed integrally with the inner shaft as a one piece configuration.

5. The inner shaft according to claim 1, wherein at least one spacer element is dome-shaped and at least one spacer element is bead-shaped, wherein the bead-shaped spacer element extends over a larger longitudinal portion along the inner shaft in comparison with the dome-shaped spacer element.

6. The inner shaft according to claim 5, wherein two bead-shaped spacer elements located opposite in the cross section are provided, wherein the dome-shaped spacer element is arranged centrally with respect thereto.

7. The inner shaft according to claim 1, wherein the inner shaft is configured, in at least one region, for contacting at least one working tool, wherein the at least one region is configured to guide the working tool so that the working tool can be guided at a distance from the outer shaft due to the shape configuration of the cross section at a predefined position.

8. The inner shaft according to claim 1, wherein the inner shaft forms a working channel configured for guiding and/or fixing a working tool, the working channel being arranged within a cross section of the inner shaft.

9. The inner shaft according to claim 7, wherein the spacer elements and the at least one region for contacting the at least one working tool are arranged adjacent to each other on the inner shaft.

10. The inner shaft according to claim 1, wherein the outer surface is concave in portions and convex in portions in different peripheral regions.

11. The inner shaft according to claim 1, wherein a wall thickness of the inner shaft is, at least in portions, 0.1 mm to 0.2 mm.

12. A method for producing an inner shaft for a working element of a resectoscope, the method comprising:
forming the inner shaft with an inner surface and an outer surface to comprise: a distal end portion; and at least three spacer elements which are arranged on the outer surface of the inner shaft, wherein the spacer elements are distributed at different positions in a region of the distal end portion such that the inner shaft can be guided and/or placed within an outer shaft at a distance from the outer shaft in a predefined way at least in the distal end portion, wherein the configuration of the inner shaft is produced by erosion.

13. The method according to claim 12, wherein at least one spacer element of the at least three spacer elements is formed by an embossing process.

14. A resectoscope comprising:
an outer shaft;
an inner shaft comprising:
a distal end portion; and
at least three spacer elements which are arranged on an outer surface of the inner shaft, wherein the spacer elements are distributed at different positions in a region of the distal end portion such that the inner shaft can be guided and/or placed within the outer shaft at a distance from the outer shaft in a predefined way at least in the distal end portion, wherein the inner shaft is guided in the outer shaft in such that, in the region of a distal end portion, a lowering of a working tool, disposed adjacent to the inner shaft, relative to the outer shaft is prevented by the spacer elements.

15. The resectoscope according to claim 14, further comprising the working tool, wherein the inner shaft is configured such that the working tool is positioned and guided so as to be returned after moving out, without contact with the outer shaft.

16. The inner shaft according to claim 1, wherein the inner shaft is configured with an inner surface and the outer surface and the outer surface is configured to be guided in the outer shaft such that, in the region of a distal end portion, a lowering of a working tool, disposed adjacent to the inner shaft, relative to the outer shaft is prevented by the spacer elements.

17. The resectoscope according to claim 14, wherein the spacer elements are arranged on the outer surface of the inner shaft at different peripheral positions and/or at different positions along a longitudinal axis of the inner shaft and are formed integrally with the inner shaft as a one piece configuration and wherein at least one spacer element is dome-shaped and at least one spacer element is bead-shaped, wherein the bead-shaped spacer element extends over a larger longitudinal portion along the inner shaft in comparison with the dome-shaped spacer element.

18. The resectoscope according to claim 14, wherein the inner shaft is configured, in at least one region, for contacting the at least one working tool, wherein the at least one region is configured to guide the working tool so that the working tool can be guided at a distance from the outer shaft due to the shape configuration of the cross section at a predefined position.

19. The resectoscope according to claim 18, wherein the inner surface of the inner shaft forms a working channel configured for guiding and/or fixing another working tool, the working channel being arranged within a cross section of the inner shaft.

20. The resectoscope according to claim 18, wherein the spacer elements and the at least one region for contacting the at least one working tool are arranged adjacent to each other on the inner shaft.
